(19) United States
(12) Patent Application Publication
(10) Pub. No.: US 2026/0053337 A1
(43) Pub. Date: Feb. 26, 2026

(54) METHOD AND APPARATUS FOR MANIPULATING A CONTROL WIRE OF A MEDICAL DEVICE

(71) Applicant: TANGENT ENDOSCOPY, LLC, Camden, DE (US)

(72) Inventors: Scott Richard ARP, Miami, FL (US); Berry LAMY, Boynton Beach, FL (US); Angel MENDOZA, Tamarac, FL (US)

(21) Appl. No.: 19/303,779

(22) Filed: Aug. 19, 2025

Related U.S. Application Data
(60) Provisional application No. 63/686,063, filed on Aug. 22, 2024.

(51) Int. Cl. A61B 1/005

(52) U.S. Cl. CPC A61B 1/0057

(57) ABSTRACT

A method and apparatus for manipulating a control wire in a medical device comprising a control wire take-up wheel being rotatable to provide coarse control wire manipulation and a ratchet assembly, coupled to the control wire take-up wheel and coupled to the control wire, to provide fine control wire manipulation.

### RELATED APPLICATION

This application claims benefit to Provisional Patent Application Ser. No. 63/686,063 filed 22 Aug. 2024 entitled “Method and Apparatus for Manipulating a Control Wire of a Medical Device,” which is hereby incorporated herein by reference in its entirety.

### BACKGROUND

### Field

Embodiments of the present invention generally relate to medical devices and, in particular, to a method and apparatus for manipulating a control wire of a medical device.

### Description of the Related Art

A typical medical device, such as an endoscope, comprises an actuator and a substantially hollow, flexible shaft extending from the actuator. The actuator manipulates at least one control wire that extends, along the inside of the shaft, from the actuator to a distal end of the shaft. The actuator generally comprises at least one take-up wheel coupled to a control knob or slider. The at least one control wire is attached to the take-up wheel. As the knob or slider is manually manipulated, the take-up wheel rotates on an axis and more or less control wire wraps around the wheel. As the control wire is taken-up by the wheel, the distal end of the flexible shaft is curved in a specific direction depending on the attachment point of the distal end of the wire to the flexible shaft.

If the medical device comprises two wires, the wires are attached to opposite sides of the wheel and manipulation of the actuator moves the distal end of the shaft left or right, or up or down. If the medical device comprises four wires. The wires are attached to two independently controllable take-up wheels and manipulation of the actuator moves the distal end of the shaft left, right, up or down, i.e., three-dimensional manipulation.

As the actuator is manipulated, the wire that is tensioned by the take-up wheel moves the distal end in a particular direction. The wires that are not tensioned will have slack in them. Upon changing direction of the manipulator, the slack must be taken-up before the distal end of the shaft will move. Such slack causes a lack of end motion for a time period until the slack is taken-up. This slack induced hysteresis can result in inaccurate positioning of the endoscope distal end and incorrect positioning of a medical instrument located at the distal end.

In addition, positioning an endoscope using the knobs or thumb slider can be inaccurate and cause errors in positioning.

Therefore, there is a need for an improved method and apparatus for manipulating a control wire of a medical device.

### SUMMARY

A method and apparatus for manipulating a control wire of a medical device is provided substantially as shown in and/or described in connection with at least one of the figures, as set forth more completely in the claims.

Various features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

So that the manner in which the various features of the present invention can be understood in detail, a particular description of the invention, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 depicts a perspective view of a medical device in accordance with at least one embodiment of the invention;

FIG. 2 depicts a top view of an actuator of the medical device of FIG. 1 in accordance with at least one embodiment of the invention;

FIG. 3 depicts a perspective view of an actuator of FIG. 2 in accordance with at least one embodiment of the invention;

FIG. 4 depicts a side plan view of the actuator of FIG. 2 having a rachet assembly in a first position in accordance with at least one embodiment of the invention;

FIG. 5 depicts a side plan view of the actuator of FIG. 2 having the rachet assembly in a second position in accordance with at least one embodiment of the invention;

FIG. 6 depicts an exploded view of the rachet assembly of the actuator of FIG. 2 in accordance with at least one embodiment of the invention;

FIG. 7 depicts a perspective view of a rachet adjuster in accordance with at least one embodiment of the invention;

FIG. 8 depicts a side cross-sectional view of an endoscope shaft distal end in accordance with at least one embodiment of the invention;

FIG. 9 depicts an end cross-sectional view along lines 9-9 in FIG. 8 of the endoscope shaft distal end in accordance with at least one embodiment of the invention;

FIG. 10 depicts a perspective view of a rachet assembly in accordance with at least one alternative embodiment of the invention;

FIG. 11 depicts an exploded view of the rachet assembly of FIG. 10 in accordance with at least one alternative embodiment of the invention; and

FIG. 12 depicts a perspective view of a rachet assembly without the cover plate in accordance with at least one alternative embodiment of the invention.

### DETAILED DESCRIPTION

Embodiments of the present invention include a method and apparatus for manipulating at least one control wire of a medical device, such as an endoscope. The medical device comprises an actuator coupled to a substantially hollow, flexible shaft. The actuator is coupled to at least one control wire that extends internal of the shaft from the actuator to a location near a distal end (or tip) of the shaft. Manipulation of the actuator pulls on the control wire and causes the distal end of the shaft to move in a specific direction. In one embodiment of the invention, the actuator comprises a coarse control mechanism and a fine control mechanism. The coarse control mechanism comprises at least one knob and at least one slider, where each controls at least one wire take-up wheel. Manipulating the knob or slider rotates the take-up wheel to wrap a portion of the control wire around the take-up wheel. The fine control mechanism comprises a ratchet assembly to accurately adjust a control wire that has been coarsely positioned by the coarse control mechanism. The ratchet assembly maintains continuous tension upon the at least one control wire and provides very accurate manipulation of the at least one control wire. Additionally, the ratchet assembly improves the movement responsiveness of the distal end of the endoscope by removing slack from the control wire. In a specific embodiment, the ratchet assembly comprises a ratchet adjuster coupled to a control wire and interacting with a rachet teeth attached to the take-up wheel. The coarse control mechanism rotates the ratchet teeth and ratchet adjuster to a coarse position, then the ratchet adjuster is manipulated to accurately position the control wire.

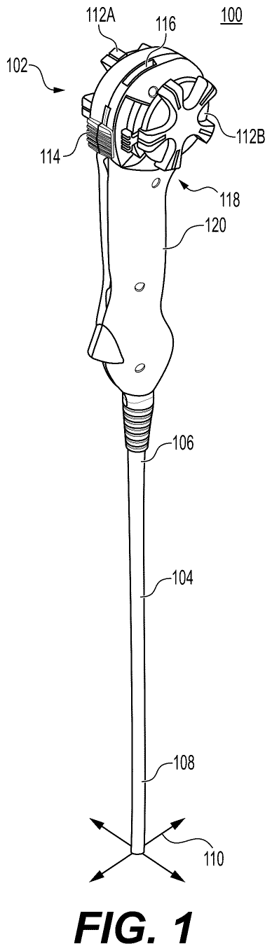

FIG. 1 depicts a perspective view of a medical device 100 (e.g., an endoscope) in accordance with at least one embodiment of the invention. The medical device 100 comprises an actuator 102 within a housing 120 coupled to a hollow flexible shaft 104. The shaft 104 comprises a proximal end 106 coupled to the housing 120 and a distal end 108 (also referred to as a tip) that is directionally manipulated by the actuator 102 (as indicated by arrows 110). In one embodiment, the actuator 102 comprises coarse control mechanism 118 formed of at least one knob 112 (shown as two knobs 112A and 112B) and/or sliders 114 that are manually manipulated in a coarse manner to move the distal end 108 of the shaft 104. The housing 120 is shaped to be held in a medical professional's hand while the knob 112 and/or slider 114 are manipulated. In addition, a fine control mechanism 116 utilizes a ratchet assembly to finely adjust the control wires as described in detail below.

In other embodiments, the actuator 102 comprises automated manipulators such as stepper motors, solenoids, and the like (not shown) that can be electronically manipulated to manipulate the coarse and fine control mechanisms to move the distal end 108 of the shaft 104.

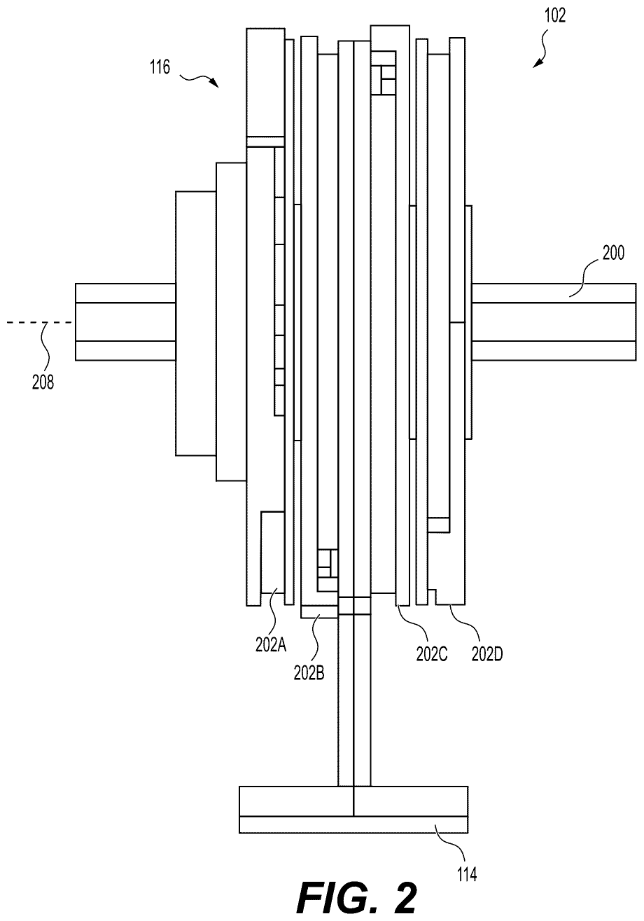

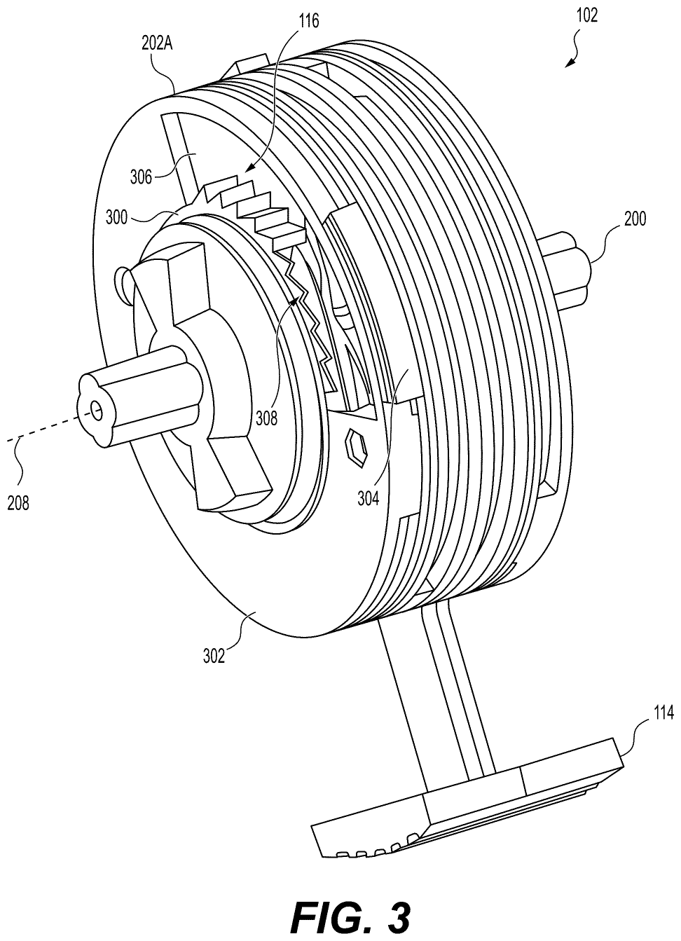

FIG. 2 depicts top view of the actuator 102 (with the housing removed) of FIG. 1 in accordance with at least one embodiment of the invention. The actuator 102 comprises at least one take-up wheel 202 (four are shown as 202A, 202B, 202C, 202D) that is manipulated by either a knob (via shaft 200), slider 114 or electronic manipulators (not shown). At least one control wire (not shown in this view) is coupled to at least one take-up wheel 202 such that rotation of the wheel 202 wraps or unwraps a portion of a control wire around a portion of the wheel 202. The fine control mechanism 116 is formed as part of each take-up wheel 202. A detailed description of the fine control mechanism 116 structure and operation is provided with respect to FIGS. 3 through 6 below.

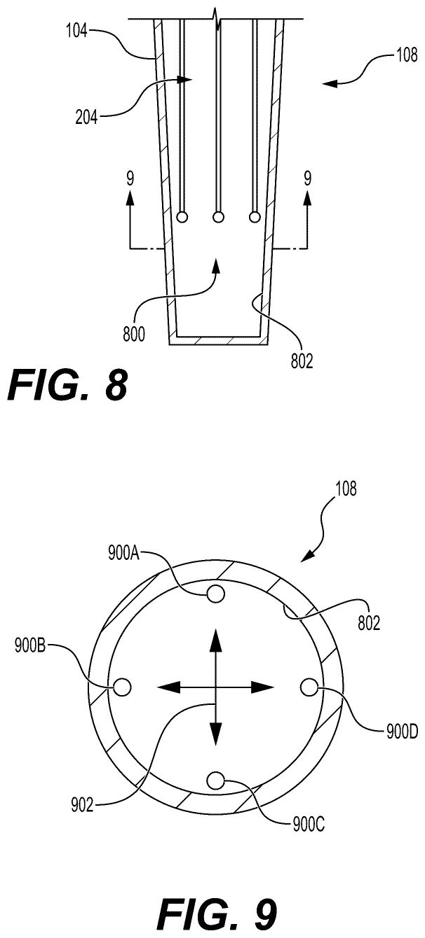

The take-up wheel 202 rotates about an axis 208. The at least one control wire is attached to a portion (e.g., within the fine control mechanism 116) of each of the wheels 202. As the wheel 202 is rotated, the at least one control wire linearly moves as the wire wraps or unwraps onto the wheel 202. In an embodiment where the tip may move in three dimensions, four wires are used with two wires coupled to two sets of separate and independently rotatable take-up wheels. The take-up wheels are stacked adjacent each other and coupled together in pairs such that each pair independently rotates about the same axis. In the depicted embodiment, take-up wheels 202A and 202D are coupled to the shaft 200 and are coarsely manipulated in one plane by the knobs 112. Additionally, take-up wheels 202B and 202C are coupled together and to the slider 114 for coarse manipulation in a second plane orthogonal to the first plane. See the description of the wire attachment within the shaft 104 with respect to FIGS. 8 and 9 below.

FIG. 3 depicts a housing removed, perspective view of the actuator 102 of FIG. 2 in accordance with at least one embodiment of the invention. In an embodiment for three-dimensional tip movement, the actuator 102 comprises four stacked take-up wheels 202A, 202B, 202C, 202D having a common rotational axis 208 to facilitate independent manipulation of each of four control wires (not shown). In the depicted embodiment, wheel 202B and 202C are manipulated by the slider 114, while wheels 202A and 202D are manipulated by knobs 112A and 112B (shown in FIG. 1) via shaft 200. In some embodiments, the knobs 112A and 112B are on a common axle with the wheels 202A and 202B such that both knobs turn the wheels simultaneously. In other embodiments, the knobs and wheels may operate independently. As each wheel 202, is rotated, the control wires move linearly to move the distal end of the shaft left, right, up and/or down.

To facilitate fine control of the wires, each take-up wheel 202 includes a fine control mechanism 116. The fine control mechanism 116 comprises a rachet assembly 308 having ratchet teeth 300 and a ratchet adjuster 304. The ratchet teeth 300 are mounted to a planar side 306 of the take-up wheel 202 and the ratchet adjuster 304 is held in place to interact with the ratchet teeth 300 by a cover 302. The proximal end of the control wire (not shown) is coupled to the ratchet adjuster 304. In this manner, the ratchet assembly 308 moves with the rotation of the take-up wheel 200 to facilitate coarse positioning of the wire. Subsequent manipulation of the ratchet adjuster 304 provides fine control of the wire positioning.

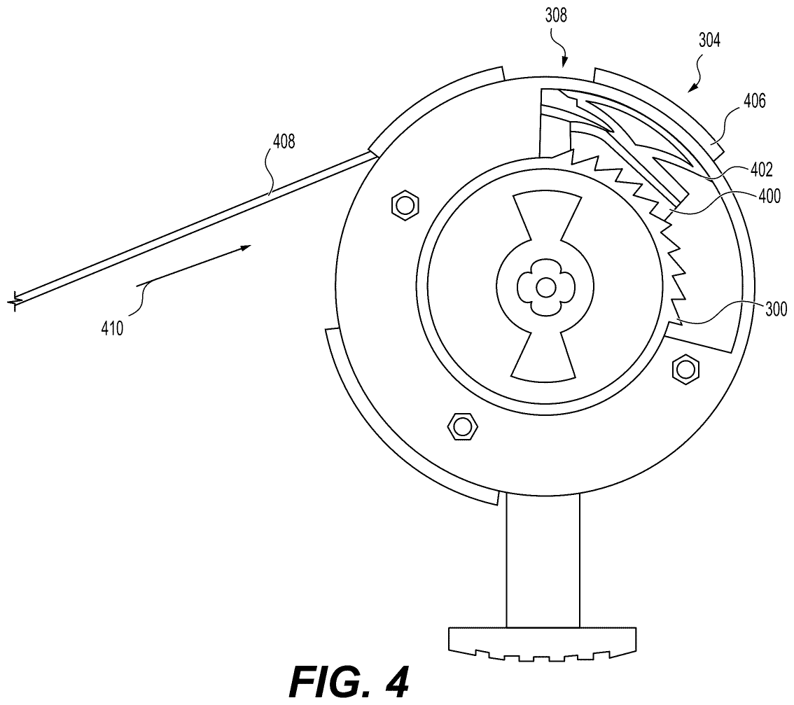

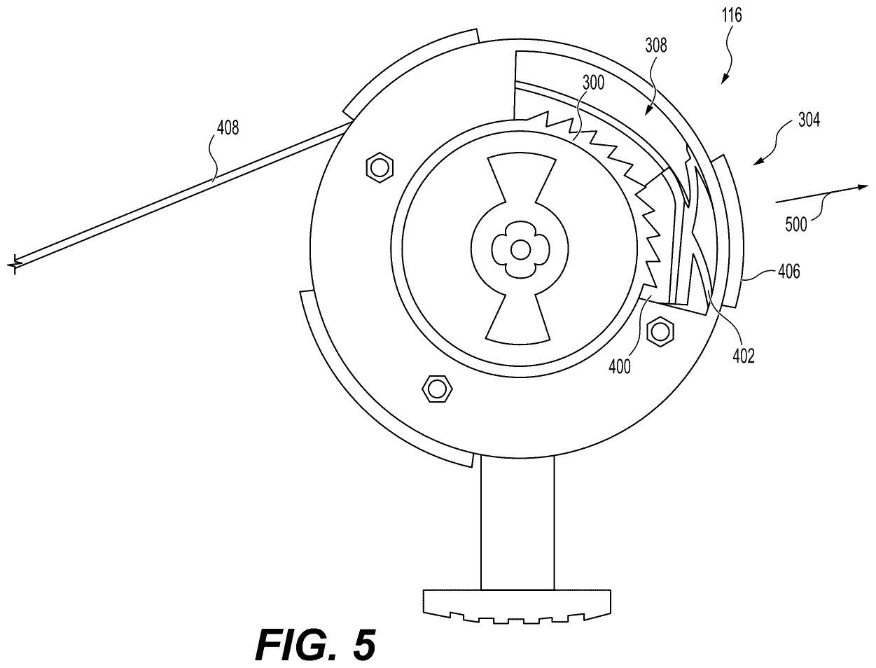

FIG. 4 depicts a housing removed, side view of the actuator 100 of FIG. 2 with the ratchet adjuster 304 in a first position in accordance with at least one embodiment of the invention. FIG. 5 depicts a housing removed, side view of the actuator 100 of FIG. 2 with the ratchet adjuster 304 in a second position in accordance with at least one embodiment of the invention.

In FIG. 4, the rachet assembly 308 is positioned fully forward. The ratchet assembly 308 comprises the take-up wheel ratchet teeth 300 and the ratchet adjuster 304. The ratchet adjuster 304 comprises at least one adjuster tooth 400 (e.g., a plurality of adjuster teeth are shown), a bias member 402 and a position controller 406. The bias member 402 biases the adjuster teeth 400 against the take-up wheel teeth 300. By moving the position controller 406 from the first position (FIG. 4) towards the second position (FIG. 5), the control wire 408 is moved inward toward the actuator 102 (arrow 410) to exert tension on the wire. The length of the teeth 300/400 determine the granularity of wire adjustment achieved by the fine control mechanism 116 (i.e., smaller teeth create finer control). The position controller 406 may be pulled outward (arrow 500) to release (disengage) the adjuster teeth 400 from the take-up wheel ratchet teeth 300 to enable the adjuster 308 to be moved toward the first position from the second position. In this manner, the position controller 406 controls the engagement and disengagement of the adjuster teeth 400 with the take-up wheel ratchet teeth 300 and controls the position of the adjuster teeth 400 relative to the take-up wheel ratchet teeth 300.

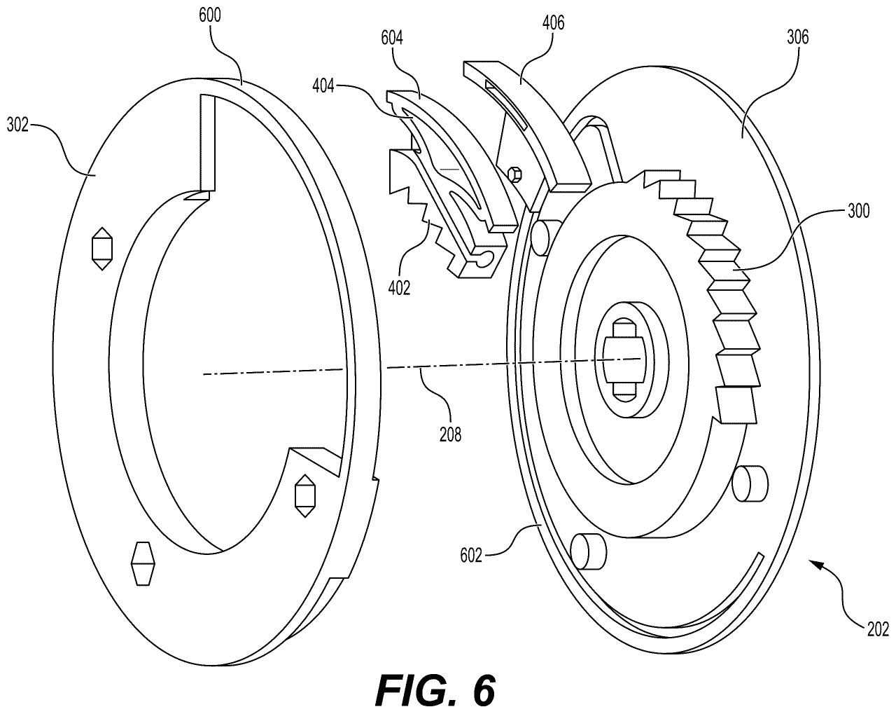

FIG. 6 depicts an exploded view of a single take-up wheel 202 with a ratchet assembly 308 in accordance with at least one embodiment of the invention. The take-up wheel 202 has the take-up wheel ratchet teeth 300 formed or attached to a front surface 306 of the wheel 202. The teeth 300 are formed in an arcuate pattern on a radius from the rotation axis 208. Extending from the outer edge of surface 306 is a flange 602 along part of the circumference of the wheel 202. This flange 602 forms a surface upon which the control wire (not shown) lies and is taken up as the wheel 202 is rotated during coarse control. The ratchet assembly 308 further comprises the position controller 406, the bias member 402 and the adjuster teeth 400. In one embodiment, the bias member 402 is a leaf spring. The ratchet assembly 308 also comprises a cover 302 that attaches to the surface 306 of the wheel 202. The cover has a flange 600 at the circumferential edge and extending from the surface facing the wheel towards the wheel surface 306. When assembled using fasteners, the flange 600 forms a gap between the flange 600 and the wheel surface 306 in which the position controller 406 is captured and retained such that the controller 406 may slide forward and backwards to control the wire position. The position controller 406 is attached to the bias member 402 such that when the controller 406 is pulled outward the bias member 404 is compressed and the teeth 300 disengage from the teeth 400. The bias member 404 comprises an arcuate shaped top surface 604 that matches the radius curve of the flange 600 such that the top surface 604 slides along the inner surface of the flange 600 when the adjuster 304 is moved.

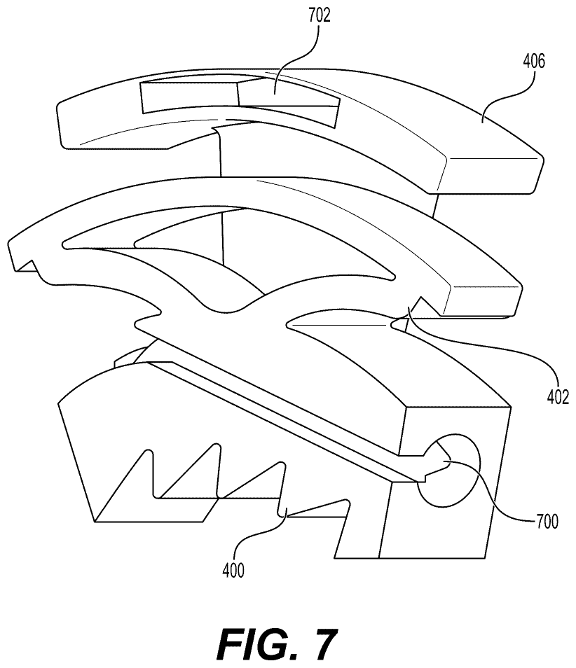

FIG. 7 depicts a perspective view of the rachet adjuster 304 in accordance with at least one embodiment of the invention. The adjuster 304 comprises a position controller 406, a bias member 402 and adjuster teeth 400. The adjuster 304 also comprises a slot 700 where a control wire end (proximal end) is attached typically by a crimp ball at the end of the wire being lodged in the slot 700. The position controller 406 is attached to the adjuster teeth 400 such that pulling upward on the controller 406 compresses the bias member 402. In one embodiment, the bias member 402 is a leaf spring (as shown). In other embodiments, the bias member 402 may be fabricated of various resilient materials and/or structures that bias the teeth 400 into contact with the take-up wheel teeth (300 in FIG. 3). To facilitate pulling the controller 406 upwards, a slot or hole 702 may be provided in the controller 406 such that a tool can be inserted and used as a handle to pull the controller 406 outward while allowing the controller 406 to rotate on the actuator when the tool/handle is not needed.

FIG. 8 depicts a side cross-sectional view of the endoscope shaft distal end 108 in accordance with at least one embodiment of the invention. FIG. 9 depicts an end cross-sectional view along lines 9-9 in FIG. 8 of the endoscope shaft distal end 108 in accordance with at least one embodiment of the invention. The distal end 108 forms the tip of the hollow, flexible shaft 104 in FIG. 1. Within the shaft, the ends 800 of the control wires 204 are attached to the inside surface 802 of the shaft 104. As shown in FIG. 9, for a four-wire endoscope, the wires 204 are attached at 90-degree locations 900A, 900B, 900C, and 900D around the circumference of the inner surface 802 of the shaft. Linear motion of the wires 204 causes the tip to be moved in a particular direct represented by the arrows 902.

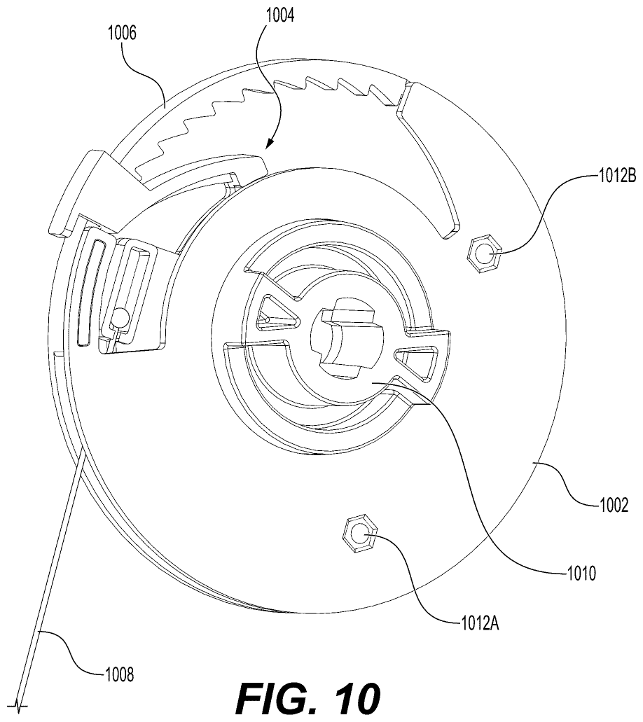

FIG. 10 depicts a perspective view of a rachet assembly 1000 in accordance with at least one alternative embodiment of the invention. The ratchet assembly 1000 comprises a cover plate 1002, a ratchet adjuster 1004, and a wheel 1006. The ratchet adjuster 1004 is captured between the cover plate 1002 and the wheel 1006 such that the ratchet adjuster 1004 may rotate and manipulate the control wire 1008. Central to the wheel 1006 is a hub 1010 that is configured to couple to the axle 200 of the actuator 102 of FIGS. 1 and 2. The cover plate 1002 may be attached to the wheel 1006 via screws, bolts, pins, rivets, adhesive and the like at, for example, points 1012A and 1012B.

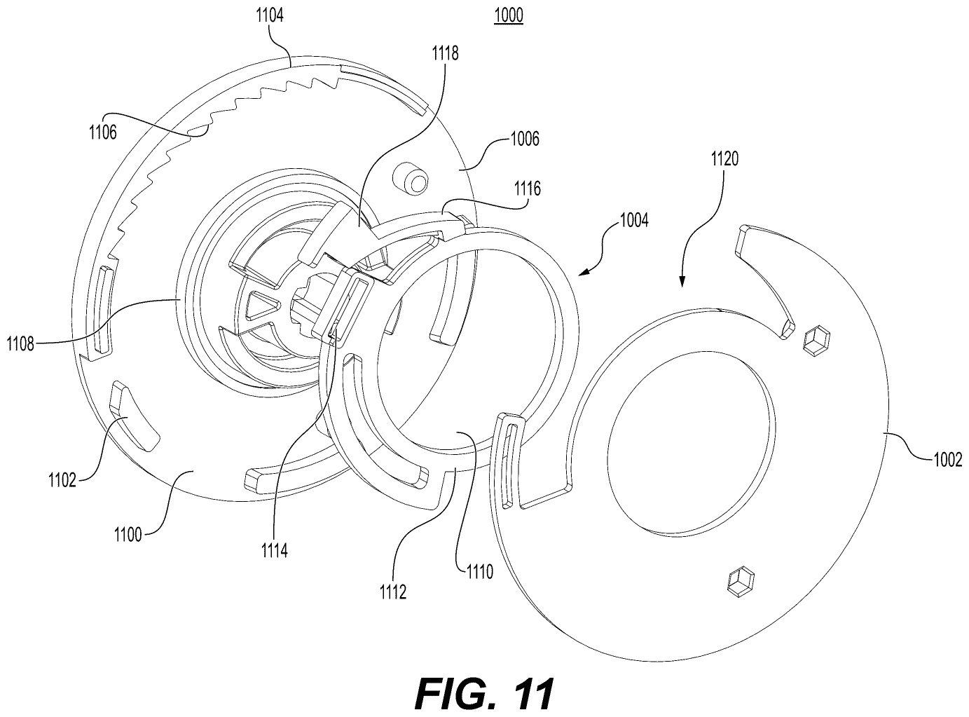

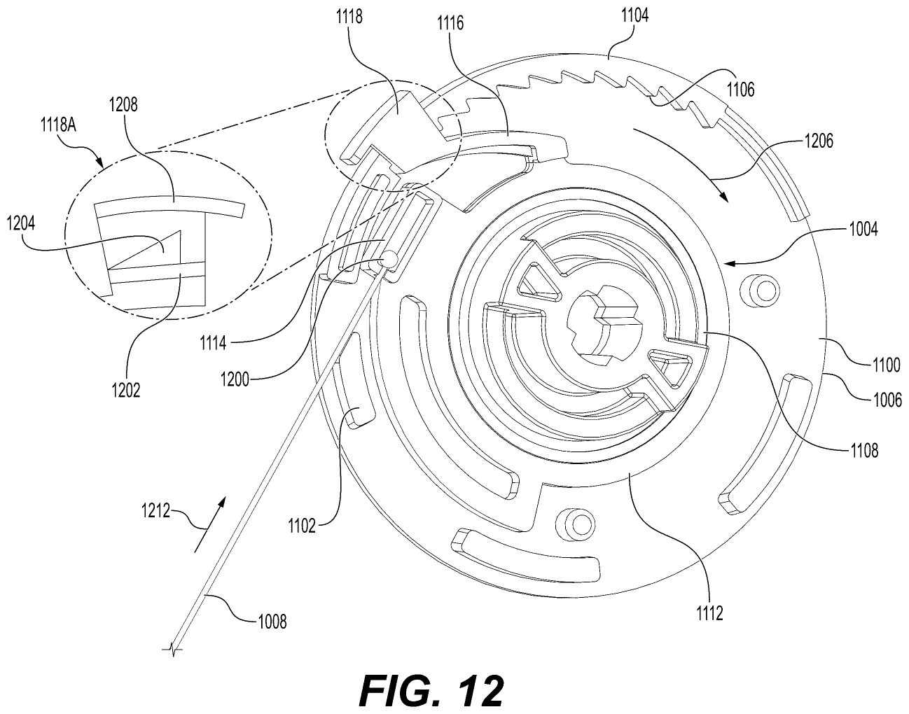

FIG. 11 depicts an exploded view of the rachet assembly 1000 of FIG. 10 in accordance with at least one alternative embodiment of the invention. The take-up wheel 1006 comprises a planar portion 1100, a wire guide 1102, take-up wheel ratchet 1104 and a circular flange 1108. The ratchet 1104 is arcuate in shape and comprises a plurality of ratchet teeth 1106 on the inner surface of the ratchet 1104. The flange 1108 forms a hub upon which the ratchet adjuster 1004 rotates. The ratchet adjuster 1004 comprises a ring member 1112 that defines an opening 1110 that circumscribes the flange 1108. The ring member 1112 supports a control wire anchor 1114 and a cantilever 1116 that couples to a position controller 1118 at the distal end of the cantilever 1116. The cantilever 1116 (a form of bias member) biases the position controller 1118 toward the ratchet teeth 1106. The cantilever 1116 is formed from a resilient material (e.g., plastic) to enable the position controller 1118 to be depressed inward to disengage the position controller 1118 from the teeth 1106 as described with respect to FIG. 12 below.

The cover plate 1002 comprises an open area 1120 that permits the ratchet adjuster 1004 to move back and forth while rotating the ring member 1112 about the flange 1108. The operation of the ratchet assembly is more fully described with respect to FIG. 12.

FIG. 12 depicts a perspective view of a rachet assembly 1000 without the cover plate 1002 in accordance with at least one alternative embodiment of the invention. FIG. 12 also depicts a detailed view of the position controller 1118 (rotated 180 degrees with respect to the view in FIG. 12). The control wire 1008 is coupled to the control wire anchor 1114 via, for example, a ferrule or ball 1200. Other means of retaining the end of the control wire 1008 into the anchor 1114 may be used.

The position controller 1118 comprises a finger pad 1208, a vertical portion 1210, a ratchet support 1202 and an adjuster tooth 1204. The finger pad 1208 is formed atop the vertical portion 1210 and slightly arcuate in shape. The ratchet support 1202 extends perpendicularly from the vertical portion 1210 and supports the ratchet tooth 1204. In the depicted embodiment, there is a single tooth 1204. In other embodiments, a plurality of teeth may be formed on the support 1202. In other embodiments, the tooth 1204 may be a post or other feature that interacts with the ratchet teeth to releasably maintain the position of the position controller 1118.

In operation, a user places a finger (e.g., index finger or thumb) upon the finger pad 1208 and moves the ratchet controller 1118 in the direction of the arrow 1206. The ratchet adjuster 1004 rotates about the flange 1108 while the tooth 1204 slides past the ratchet teeth 1106 of the ratchet 1104. As the ratchet adjuster 1004 rotates the control wire 1008 is pulled past wire guide 1102 in a linear manner (arrow 1212). The length of the ratchet teeth 1106 defines the granularity of the fine wire control (i.e., smaller teeth provide finer control).

To rotate the ratchet adjuster 1004 in an opposite direction to arrow 1206, a user depresses the finger pad 1208 of the position controller 1118 which forces the cantilever 1116 inward to release the tooth 1204 from the rachet teeth 1106. The control wire 1008 then moves in the opposite direction to arrow 1212. Upon reaching the desired position, the user releases the finger pad 1208 and the cantilever 1116 biases the adjuster tooth 1204 into contact with the ratchet teeth 1106 to lock the ratchet assembly 1000 into position. Consequently, the ratchet assembly 1000 provides fine positioning control of the control wire 1008.

Here multiple examples have been given to illustrate various features and are not intended to be so limiting. Any one or more of the features may not be limited to the particular examples presented herein, regardless of any order, combination, or connections described. In fact, it should be understood that any combination of the features and/or elements described by way of example above are contemplated, including any variation or modification which is not enumerated, but capable of achieving the same. Unless otherwise stated, any one or more of the features may be combined in any order.

As above, figures are presented herein for illustrative purposes and are not meant to impose any structural limitations, unless otherwise specified. Various modifications to any of the structures shown in the figures are contemplated to be within the scope of the invention presented herein. The invention is not intended to be limited to any scope of claim language.

Where conditional language is used, including, but not limited to, “can,” “could,” “may” or “might,” it should be understood that the associated features or elements are not required. As such, where conditional language is used, the elements and/or features should be understood as being optionally present in at least some examples, and not necessarily conditioned upon anything, unless otherwise specified.

Where lists are enumerated in the alternative or conjunctive (e.g., one or more of A, B, and/or C), unless stated otherwise, it is understood to include one or more of each element, including any one or more combinations of any number of the enumerated elements (e.g. A, AB, AC, ABC, ABB, etc.). When “and/or” is used, it should be understood that the elements may be joined in the alternative or conjunctive.

While the foregoing is directed to embodiments of the present invention, other and further embodiments of the invention may be devised without departing from the basic scope thereof, and the scope thereof is determined by the claims that follow.

1. Apparatus for manipulating a control wire of a medical device comprising:
a control wire take-up wheel being rotatable to provide coarse control wire manipulation; and
a ratchet assembly, coupled to the control wire take-up wheel and coupled to the control wire, to provide fine control wire manipulation.

2. The apparatus of claim 1 wherein the ratchet assembly further comprises:
ratchet adjuster, coupled to the control wire, having at least one adjuster tooth adapted to interact with take-up wheel ratchet teeth that are attached to the control wire take-up wheel to provide fine control wire manipulation.

3. The apparatus of claim 2 wherein the ratchet adjuster further comprises a bias member to bias the at least one adjuster tooth against the take-up wheel ratchet teeth.

4. The apparatus of claim 3 wherein the ratchet adjuster further comprises a position controller, coupled to the at least one adjuster tooth, to control a position of the at least one tooth relative to the take-up wheel ratchet teeth and control engaging and disengaging of the at least one adjuster tooth with the take-up wheel ratchet teeth.

5. The apparatus of claim 2 wherein the length of the teeth within the take-up wheel ratchet teeth and the at least one adjuster tooth defines the granularity of fine control wire manipulation.

6. The apparatus of claim 1 further comprising a plurality of control wire take-up wheels, where each control wire take-up wheel in the plurality of control wire take-up wheels is associated with a ratchet assembly.

7. The apparatus of claim 6 wherein the ratchet assembly further comprising:
ratchet adjuster, coupled to the control wire, having at least one adjuster tooth adapted to interact with take-up wheel ratchet teeth that are attached to an associated control wire take-up wheel to provide fine control wire manipulation.

8. The apparatus of claim 7 wherein the ratchet adjuster further comprises a bias member to bias the at least one adjuster tooth against the take-up wheel ratchet teeth.

9. The apparatus of claim 8 wherein the ratchet adjuster further comprises a position controller, coupled to the at least one adjuster tooth, to control a position of the at least one tooth relative to the take-up wheel ratchet teeth and control engaging and disengaging of the at least one adjuster tooth with the take-up wheel ratchet teeth.

10. The apparatus of claim 7 wherein the length of the teeth within the take-up wheel ratchet teeth and the at least one adjuster tooth defines the granularity of fine control wire manipulation.

11. The apparatus of claim 1 wherein the control wire extends from the ratchet assembly to a distal end of a hollow shaft to effectuate manipulation of the distal end upon adjustment of the control wire take-up wheel and/or the ratchet assembly.

12. A method of manipulating a control wire of a medical device comprising:
rotating a control wire take-up wheel to provide coarse control wire manipulation; and
operating a ratchet assembly to provide fine control wire manipulation, where the ratchet assembly is coupled to the control wire take-up wheel and is coupled to the control wire.

13. The method of claim 12 wherein rotating the control wire take-up wheel further comprises manipulating a knob and/or slider to cause the rotation of the control wire take-up wheel.

14. The method of claim 12 wherein operating a ratchet assembly further comprises:
manipulating a position controller to cause at least one adjuster tooth to engage or disengage take-up wheel ratchet teeth, wherein the take-up wheel ratchet teeth are attached to the control wire take-up wheel.

15. The method of claim 14 wherein manipulating the position controller compresses a bias member to disengage the at least one adjuster tooth from the take-up wheel ratchet teeth, wherein the bias member is adapted to bias the at least one adjuster tooth to engage the take-up wheel ratchet member.

16. The method of claim 15 wherein a length of the at least one adjuster tooth and a length of teeth in the take-up wheel ratchet teeth defines a granularity of the fine control wire adjustment.

17. The method of claim 15 wherein rotating the take-up wheel coarsely positions the take-up wheel ratchet teeth and operating the ratchet assembly moves the at least one adjuster tooth relative to the coarsely positioned take-up wheel ratchet teeth to provide fine control wire positioning.

18. The method of claim 17 wherein the control wire extends from the ratchet assembly to a distal end of a hollow shaft to effectuate manipulation of the distal end upon adjustment of the control wire take-up wheel and/or the ratchet assembly.

19. Apparatus for manipulating a plurality of control wires of a medical device comprising:
an actuator within a housing comprising:
a plurality of control wire take-up wheels, each being independently rotatable to provide coarse control wire manipulation; and
a plurality of ratchet assemblies, where each ratchet assembly is coupled to an associated one of the control wire take-up wheels in the plurality of control wire take-up wheels and each ratchet assembly is coupled to one of the control wires, to provide fine control wire manipulation; and
a hollow shaft, having a proximal end and a distal end, where the plurality of control wires extend through the hollow shaft and are coupled to the inside of the hollow shaft proximate the distal end.

20. The apparatus of claim 19 wherein each of the ratchet assemblies further comprises:
a ratchet adjuster, coupled to one of the control wires, comprising:
at least one adjuster tooth adapted to interact with take-up wheel ratchet teeth that are attached to the associated take-up wheel to provide fine control wire manipulation.
a bias member to bias the at least one adjuster tooth against the take-up wheel ratchet teeth; and
a position controller, coupled to the at least one adjuster tooth, to control a position of the at least one tooth relative to the take-up wheel ratchet teeth and control engaging and disengaging of the at least one adjuster tooth with the take-up wheel ratchet teeth of the associated one of the take-up wheels.
